(19) United States
(12) Patent Application Publication
(10) Pub. No.: US 2008/0118475 A1
(43) Pub. Date: May 22, 2008

(54) Model System for Testing the Efficacy of Pharmaceutical Preparation on Inflammatory Processes in the Nervous System

(75) Inventors: Frank Striggow, Gerwisch (DE); Till Mack, Magdeburg (DE); Peter Roehnert, Magdeburg (DE)

Correspondence Address: GREENBLUM & BERNSTEIN, P.L.C., 1950 ROLAND CLARKE PLACE, RESTON, VA, 20191, US

(73) Assignee: KEYNEUROTEK AG, Magdeburg (DE)

(21) Appl. No.: 11/664,584

(22) PCT Filed: Sep. 22, 2005
(86) PCT No.: PCT/EP05/10281

§ 371 Date: Jul. 3, 2007

(30) Foreign Application Priority Data
Oct. 5, 2004 (DE) 10 2004 048 462.7

(51) Int. Cl. A61K 35/30; C12N 5/06; C12N 5/08

(52) U.S. Cl. 424/93.7; 435/325; 435/368; 435/354; 435/353

(57) ABSTRACT

Live cells obtained from a mammalian tissue or organ. The cells are maintained ex vivo on a porous membrane having a pore size of ≧0.02 μm at a physiologically acceptable pH, in the presence of a culture gas, in culture and in a culture medium. The inter-neighbour relationships and the signal transduction between the cells are retained.

The invention relates to mammalian cells, their recovery and ex-vivo cultivation and their use as a model system for testing the efficacy of pharmaceutical preparations and, especially, of active ingredients in pharmaceutical preparations, used for therapy and prevention of chronic and acuté neuro-pathological conditions and, especially, in respect of their neuro-protective and inflammation-modulating effectiveness. As a functional test system with a complex structural architecture, organo-typical, cultured hippocampal serial slices can be used, preferably from the rat, which with a higher throughput of test substances permit a similarly accurate predictability in respect of the effect of the substances as is possible with the much more time-consuming and costly use of whole animal models.

Inflammations provide a fundamental basis for the body's auto-protection behaviour. Through inflammation processes following injury or external assault, as a component of the immune response, the body attempts to re-establish or retain its homeostasis. Thus, inflammation in general promotes an improvement in the body's status and, specifically, improvement in the targeted tissues. Excessive or long-lasting inflammations, on the other hand, can also lead to injury in those targeted tissues.

The immune status of the brain is very precisely regulated and controlled. In a healthy state, the immune response and thereby inflammatory processes are reduced to a minimum. In contrast to that, during the course of neuro-degenerative conditions (neuropathies) such, for example, as Morbus Alzheimer, Morbus Parkinson, Multiple Sclerosis, strokes and traumas, as well as neoplasms and infections, glia-cells are activated. Pro-inflammatory factors are synthesised on site, and T-lymphocytes penetrate into the effected region of the brain.

All new rodegenerative lesions, especially where these result from fibril protein deposition or are accompanied by these, lead to necrotic inflammation reactions. These comprise the already mentioned cellular changes such, for example, as activation of the micro-glia as well as liberation of pro-inflammatory factors. Should the inflammatory reaction penetrate beyond its actual target, swiftly eliminating the local cell fragments of the already perished neurons, then instead, neighbouring, actually healthy neurons will also be damaged. Taking into account the multiplicity of functions of pro-inflammatory factors, it is very difficult to determine the precise role played by them in specific (patho)-physiological situations. The altered expression of pro-inflammatory factors can either accelerate or counteract neuro-degenerative processes (Wyss-Coray, T. and Mucke, L.: Inflammation in neuro-degenerative disease—a double-edged sword; Neuron, 2002: 35, 419 to 432). Since, therefore, inflammatory processes also contribute to the healing of wounds, the selective elimination of the neuro-degenerative, (patho-) physiological processes would be a very promising therapeutic aim, and probably superior to the complete suppression of an inflammation as practiced hitherto.

Hitherto, for the most part, cell culture models and animal models have been developed as model systems for analysing the complex interactions of the component cell types in inflammation reactions in brain tissues. In animal models, the stereo-tactic injection of bacterial lipo-polysaccharides (LPS) triggers a strong immune response from the outer membrane of gram-negative bacteria as a ‘pathogen-associated molecular pattern’ into the striatum (author's unpublished data). Thereby, dispersed micro-glia cells, which function in the brain as macro-phages, are activated, that is to say they are prepared to act as phagocytes and to be stimulated into division. This disposition of phagocytic micro-glia is the most important indication of the inflammation in the brain. It is probable that an invasion of non-participating brain regions also takes place. As a result, already after only a few days, a degeneration of the tissue, including the neurons, can be observed. The density of the nerve cells is considerably reduced, and the tissue becomes progressively scarred. Apparently, there is also an invasion of immune cells from the blood via the branch canal that can be clearly discerned at the centre of the changes in the brain tissue (author's unpublished data). It can be assumed that activated, that is to say, phagocytic micro-glia are responsible for the removal of degenerating neurons. There are even indications that they are responsible for the life or death decision for neurons that are capable of surviving. In addition, they provide for the production of a large number of pro-inflammatory and neuro-toxic factors such, for example, as cytokins, fatty acid metabolites and free radicals, nitrous oxide (NO) and super oxide—for an extension of the inflammation and thus of the degeneration. The process of the neuro-inflammation can, therefore, lead to a vicious circle wherein local inflammation always also produces neuro-toxicity or neuro-degeneration which itself again spreads the inflammation reaction which itself once again leads to an extension of the neuro-degeneration. In this way, during the course of the illness, it will be completely irrelevant whether an initial neuro-degeneration or an inflammation reaction was the starting point, since this cycle continually reinforces itself without external intervention.

Individual factors in this complex pathogenesis can also be replicated in reductionist cell culture models. In this way, co-cultures of primary micro-glia cells or micro-glia-type cell lines with primary neurons become attached. Activation of the micro-glia can be achieved in this model by means of LPS or tri-methyl-tin (Golde, S., Coles, A., Lindquist, J. A., Compston, A. (2003): Decreased iNOS synthesis mediates dexamethasone-induced protection of neurons from inflammatory injury in vitro; Eur. J. Neurosci. 18: 2527 to 2537; Eskes, C., Juillerat-Jeanneret, L., Leuba, G., Honegger, P., Monnet-Tschudi, F. (2003): Involvement of micro-glia-neutron interactions in the tumor necrosis factor-alpha release, micro-glial activation, and neuro-degeneration induced by trimethyl-tin; J. Neurosci. Res. 71: 583 to 590). As a relevant selective parameter, the quantity of neuronal cell deaths, or the survival rate of neurons can be determined. Thereby, it was already possible to demonstrate that inter alia NO as the neuro-toxic factor was the cause of neuro-degeneration in the co-culture model system, which by means of a corresponding suppression of NO-production could be arrested. (see above, Golde et al., 2003).

An advantage of the cell culture model, therefore, is its simplicity and availability, which facilitates quantitative analysis and thereby the testing of potential protection agents in a relatively short time.

Serious disadvantages are that the complex synthesis of brain tissue by co-cultures can only be reproduced in a very reduced way. In addition, for these co-cultures, either primary cells from embryonic animals or cell lines can be used, whose condition and behaviour differ markedly from those of adult cells in the adult brain. Consequently, these models offer a good throughput but not a good predictability quality, which is of concern for the efficacy of the test substances in animal models as well as, later, in human patients.

The aim of the present invention was, therefore, to make available a cell system that is simple, readily available and, at short notice, quantitatively analysable, which will accurately reproduce the complex construction of brain tissue. An additional aim of the present invention was to make available cells whose structure and behaviour is comparable to those of adult brain cells, so that with such cells the results obtained from tests allow reliable predictability in respect of the efficacy of the test substances. The aim of the present invention was further to propose the uses for such cells, as well as to provide methods which make use of these cells.

Within the scope of the present invention it was found that organo-typical cultures extracted from tissues and organs of a mammal, especially from organs or tissues from the mammalian nervous system, can be maintained ex vivo as organo-typical cultures under appropriate conditions and that in cultivating them the inter-neighbour relationships important for behaviour within the cell structure as well as signal-transduction between cells are retained.

Therefore, the present invention is concerned with live cells obtained from tissues or organs from mammals, that are maintained ex vivo on porous membranes having a pore size ≧0.02 μm at a physiologically acceptable pH-value, in the presence of a culture gas, in culture, and in an appropriate culture medium, and characterised in that they have retained their inter-neighbour relationships and the signal transduction between cells. The cells of the present invention are such, for example, (but without the invention being restricted thereto) as cells extracted from mammalian nervous system tissue, especially preferably cells extracted from the mammalian hippocampus.

Preferred embodiments of the live cells claimed in the present invention are described in the subsidiary Claims 2 to 12.

Furthermore, the present invention concerns a process for obtaining live mammalian cells maintained in cultures, as described in the following text and which comprises the following stages, namely that
tissue slices are obtained by a known method from functional mammalian tissue;
the thereby obtained tissue slices are maintained in culture ex vivo on porous membranes having a pore size ≧0.02 μm at a physiologically acceptable pH-value in the presence of a culture gas in culture and in an appropriate culture medium in such a manner that the inter-neighbour relationships and the signal transduction between cells are retained.

The present invention also relates to a process for the culture of live mammalian cells maintained ex vivo, as described in the following text and comprising the following stages, wherein tissue slices that have been obtained in a known manner from functional mammalian tissue are maintained ex vivo on porous membranes having a pore size ≧0.02 μm at a physiologically acceptable pH-value in the presence of a culture gas in culture in an appropriate culture medium in such a manner that the inter-neighbour relationships and the signal transduction between cells are retained.

In addition, the present invention concerns a process for the induction of inflammatory processes on live mammalian cells maintained ex vivo, which comprises the following stages
tissue slices are obtained from functional mammalian tissue by a known method;
the thereby obtained tissue slices are maintained ex vivo on porous membranes having a pore size ≧0.02 μm at a physiologically acceptable pH-value in the presence of a culture gas in culture in an appropriate culture medium in such a manner that the inter-neighbour relationships and the signal transduction between cells are retained; and
the cells of the process are subjected to the action of at least one pro-inflammatory carrier over a transient period of time.

Preferred embodiments of this process are claimed in the subsidiary Claims 16 and 17.

The present invention further relates to a process for the induction of inflammatory processes on live mammalian cells maintained ex vivo that comprises the following stages
tissue slices are obtained from functional mammalian tissue by a known method;
the thereby obtained tissue slices are maintained ex vivo on porous membranes having a pore size ≧0.02 μm at a physiologically acceptable pH-value in the presence of a culture gas in culture in an appropriate culture medium in such a manner that the inter-neighbour relationships and the signal transduction between cells are retained; and
the cells of the process are subjected to the action of at least one inflammogen over a transient period of time.

Preferred embodiments of this process are claimed in the subsidiary Claims 19 and 20.

Preferred embodiments for all process variants of the present invention are claimed in Claims 21 to 30.

The present invention also concerns the use of live mammalian cells maintained ex vivo in culture for medical purposes, described in detail below.

The present invention also concerns the use of live mammalian cells maintained ex vivo in culture for the ex vivo investigation of inflammatory processes described in detail below, especially for testing pharmaceutical preparations, and, more especially, for testing pharmaceutical preparations in respect of their neuro-protective and inflammation-modulating activity.

Preferred embodiments of the above-mentioned uses are claimed in the Claims 34 to 40.

The present invention further concerns a process for testing the anti-inflammatory efficacy of a chemical substance, comprising the following stages:
obtaining tissue slices from functional mammalian tissue, especially from mammalian brain tissue;
ex vivo cultivation of the tissue slices obtained on porous membranes having a pore size ≧0.02 μm at a physiologically acceptable pH-value in the presence of a culture gas in culture in an appropriate culture medium in such a manner that the inter-neighbour relationships and the signal transduction between cells are retained, and with the retention of a tissue culture that comprises live mammalian cells;
application of at least one pro-inflammatory carrier onto the tissue culture;
application of at least one chemical substance whose anti-inflammatory efficacy is to be tested onto the tissue culture;
application of at least one reagent for evidence of anti-inflammatory efficacy on to the tissue culture while aiming for quantifiable evidence of reaction; and
quantification of the evidence of reaction on the basis of the product obtained from the evidence of reaction.

Preferred embodiments of the process for testing according to the present invention are claimed in the subsidiary Claims 42 to 50.

The assay developed by the inventors combines the advantages of cell-culture models with the endogenous complexity of tissue cultures that, as system communicating cells, reflect in a much better manner the biology of a damaged organ. In addition, this ex vivo model system can be transferred onto an automated platform for organo-typical cultures (e.g. Telomics Robotic™). In this manner, a considerably greater throughput of test substances is facilitated without the complexity of model systems or restricting its predictability capacity. For the first time, the automated, systematic screening of anti-inflammatory substances is as a result possible.

The term “mammalian” as used in the preceding description and in the patent claims, refers to any mammal or mammalian animal such, for example, as guinea pigs, mice, rats, dogs, cats or monkeys, and refers, in the preferred embodiments, to mice, rats or humans, and more especially preferred, refers to humans. Cells derived from humans are especially preferred because they yield the best results for the vital tissue model for the intended purposes such as those described in detail in the following text.

The mammalian cells described in the present invention, are organo-typical cultures that have been obtained from the functional tissues or organs of a mammal and that can be maintained ex vivo over several days continually maintaining their functionality. The term “functional tissue” used within the framework of the present description and in the patent claims, is understood to mean that the complex relationships between cells in the organo-typical tissue culture are maintained, which normally can only be realized in whole animal models. Such cells can be any cells from mammalian tissues or organs. Within the present invention, however, the preferred cells are extracted from the nervous system of a mammal and, even more preferred are cells from the central nervous system (CNS), cells from the spinal cord, cells from the hippocampus and cells from the cerebral cortex. Especially preferred are cells from the hippocampus and cells from the cerebral cortex because of their good suitability for the purposes of the present invention. Such cells are readily available and can be obtained by known methods. This can, for example, be undertaken, without thereby restricting the present invention, by the preparation of tissue slices with commercially available instruments such, for example, as the McIlwain Tissue Chopper. Thereby, tissue slices are obtained that have the appropriate thickness for the culture procedure, such, for example, as a thickness that ranges from 1 to 1,000 μm, preferably a thickness of 10 to 800 μm, more preferably a thickness of 100 to 600 μm such, for example, as a thickness of 350 to 450 μm and preferably a thickness of 400 μm.

The tissue slices obtained are maintained ex vivo, which is understood in the description and in the patent claims to mean that the tissue slices extracted from the living organism are maintained in such a manner so as not to restrict the vitality and functionality of the cells. It is especially surprising that, according to the present invention, it has been possible to achieve live cells that are characterised by their retention of the inter-neighbour relationships and the signal induction between cells, which, preferably, is characterised by the neuro-physiological transfer of nerve cell impulses.

In accordance with the present invention, the live cells are maintained on porous membranes that have a pore size ≧0.02 μm according to the invention. Such porous membranes are known as such to the person skilled in the art for the purposes indicated here, and they are also commercially available. In preferred embodiments of the invention such porous membranes are permeable to a known culture medium, also used in the present invention, and, in addition, more preferably to gases such, for example, as culture gas used in the culture of cells.

Still more preferably, membranes according to the invention are those that are also permeable to materials, even oligomeric or polymeric materials that are used in culture medium. These correspond, for example, to a preferred embodiment of the present invention that membranes used for the culture of mammalian cells that are maintained ex vivo are also permeable to at least one pro-inflammatory carrier and/or to at least one inflammogen such as those which are used according to the present invention. With membranes that are permeable to one or more of the mentioned substances, especially good culture results and test-results as well as high throughput rates in the process of the present invention are obtainable.

According to an especially preferred embodiment of the present invention, the membranes used have a pore size in the range of 0.02 to 10 μm, still more preferably 0.1 to 1 μm and most preferably a pore size in the range of 0.2 to 0.6 μm such, for example, as a pore size of 0.2 or 0.4 μm.

According to the present invention, the mammalian cells are maintained ex vivo in culture in a suitable culture medium. The term ‘in culture’, as used in the present description and in the patent claims, means, in this connection, that the cells are not maintained in a live organism, but, in a substantially complete condition (insofar as they are not surrounded by other, possibly similar cells), surrounded by a synthetic, substantially liquid, but possibly also solid, dissolved and/or gaseous (or gaseous dissolved) component-containing medium, that can continually supply it with the necessary or favourable components having particular functions, especially nutrients, trace elements, vitamins, the pH-regulating materials, buffers, particular supporting, liberating or modulating substances, antibiotics and other materials. Culture media that are known to the person skilled in the art can be used without limitation. Thus, for example, culture media can be used that are selected from the literature in Dulbecco's Modified Eagle's Medium (DMEM), F12, Neurobasal Medium, MEM, PRMI (RPMI) 1640, etc. Especially preferred as the culture medium, since it is well-suited or especially well-suited for the culture of the cells of the present invention, as well as for the maintenance of the cells of the present invention, for testing the cells of the present invention or for utilization of the cells of the present invention, is DMEM/F12 (as a mixture in the volume ratio of 1:1 of DMEM and F12). A culture medium can be used on its own or several culture media can be used. This is dependent as much upon a particular stage in the culture, maintenance, testing and/or utilization of the cells as on a series of several, parallel or successive process stages that can be undertaken in one or several culture media. The culture medium or media can also contain known supplementary materials which include, but are not restricted to sera (such, for example, as foetal cattle serum, cattle serum, horse serum, B27, N2), antioxidants such as ascorbic acid, vitamin E, etc.

The mammalian cells maintained ex vivo in accordance with the present invention are maintained in the culture medium at a physiologically acceptable pH-value. By ‘physiologically acceptable pH-value’ as used in the present description and in the patent claims is meant a pH-value that is tolerable to the cells from a physiological point of view, if not actually beneficial or promotional, in respect of the aims sought by the present invention. In particular, in a preferred procedure according to the present invention, the pH-value has a pH-value that corresponds to the particular cellular environment in which the cells exist naturally or in which they can be most advantageously maintained. Especially preferred in the process of the present invention, is a physiologically acceptable pH-value within the range of 6.5 to 8, still more preferably in the range of 6.9 to 7.6, and still further preferred in the range of 7.3 to 7.5 such, for example, as 7.4.

According to the present invention, the mammalian cells are maintained in the presence of a culture gas in the culture medium. As the culture gas, any gas may be considered that is known to the person skilled in the art for fumigating ex vivo-maintained mammalian cells in culture media. As examples of the gases in the presence of which mammalian cells can be maintained in a culture medium, these can be selected from the group comprising air (by which is to be understood not only the atmospheric air with its natural composition [essentially, 78.1% by vol. of nitrogen, 21.0% by vol. of oxygen, 0.9% by vol. of argon and minor quantities of carbon dioxide], but also other similarly composed gas mixtures, and, if required, also purified air [“pure air”] that is freed from undesirable components), comprising oxygen, nitrogen and carbon dioxide, and air that has been enriched with oxygen, nitrogen or carbon dioxide, etc. According to the present invention, it is preferred that the culture gas is pure air, preferably pure air containing an increased content of CO₂, more preferably pure air having a content of CO₂ within the range of 1 to 5% by vol., still more preferably pure air with a content of CO₂ within the range of 2 to 3.5% by vol. Cells that are maintained in the presence of the above-mentioned culture gases, according to the present invention, exhibit a special degree of vitality and, consequently, are excellent, stable ex vivo model systems for testing the active ingredients in pharmaceutical preparations in respect of their neuro-protective and inflammation-modulating effects.

According to a preferred embodiment of the present invention, the live mammalian cells maintained ex vivo are cells that over a transient period of time were subjected to at least one substance that is selected from the group comprising pro-inflammatory carrier and inflammogens. As the pro-inflammatory carriers, are to be understood in the present description and in the patent claims, such materials which are secreted during inflammation reactions in various tissues for the purpose of cell communication. Pro-inflammatory carriers are such as are known extensively to the person skilled in the art and be used without restriction in accordance with the present invention. The term “inflammogen”, on the contrary, is defined for the present description and in the patent claims as comprising substances such, for example, as endotoxins, which contain pathogen-associated models that are recognised by the model-recognition receptors on the cells of the immune system and thereby elicit an unequivocal response such, for example, as a response to the presence of invading micro-organisms. Inflammogens are also known as such extensively to the person skilled in the art and can be incorporated into the process of the present invention without restriction. In each case, one or more pro-inflammatory carriers or one or more inflammogens can be incorporated either individually or in groups or in larger numbers of each without restricting the process according to the present invention.

According to a preferred embodiment of the present invention, the live mammalian cells that are maintained in culture ex vivo were, over a transient period of time, strictly subjected to a substance that was selected from a group of pro-inflammatory carriers and inflammogens. Here, a still further preferred embodiment of the present invention is indicated when the pro-inflammatory carrier(s) is/are selected from the group of pro-inflammatory proteins and peptides, preferably from the group of cytokines, more preferably from the group of CSF-proteins and interferons, and still more preferably from the group the group comprising GM-CSF, G-CSF and M-CSF and IFNy, and/or, when the inflammogen(s) is/are selected, from the endotoxin group, more preferably from the group of lipo-polysaccharides (LPS), still more preferably from the group of bacterial lipo-polysaccharides. Further examples of inflammogens belonging to the endotoxin group are the peptido-glycanes, lipo-teichonic acids, mannose-rich glycanes, flagellin, pillin, non-methylated cytosine-guanine dinucleotides, bacterial DNA, N-formyl-methionine, double-branched RNA, lipo-teichinic acid/glyco-lipids/zymosane from yeast cells, phosphoryl choline and other lipids from bacterial membranes.

The term “transient period of time”, as used in the above description and in the patent claims, is intended to denote that the cells are not exposed to the named substance(s) continuously, but only temporarily. The person skilled in the art can in individual cases determine the period of contact time of the cells with the named substance(s) on the basis of individual tests, or knows on the basis of his experience the times which should form the basis of such a contact. The transient period of time in a case where several substances are used side-by-side or consecutively can be the same or can vary for different substances. The period of time can also vary depending upon the type of mammalian cell used (for example, mouse cell or human cell), or upon the type of cell used (for example, nervous system cell, cerebral cortex cell). According to the present invention, it is especially preferred if the period of transient time taken, where at least one pro-inflammatory carrier is used, is within the range of 10 minutes and 7 days, preferably within the range of 15 min. to 4 days, more preferably within the range of 30 min. to 2 days, and/or that the transient period of time taken when at least one inflammogen is used is within the range of 1 day and 14 days, preferably within the range of 1 day to 4 days, and more preferably within the range of 2 days and 3 days.

The live mammalian cells maintained in culture as described above in accordance with the present invention, are able to be used, in their preferred form, for any desired medical purpose. They are not restricted but selected by the person skilled in the art on the basis of his technical know-how and depend upon the specific suitability of the cells selected. Especially advantageously suitable are the live mammalian cells maintained in culture, that are appropriate for the above-detailed description of the ex vivo testing of inflammatory processes, especially for testing pharmaceutical preparations, and more preferably for testing pharmaceutical preparations in respect of their neuro-protective and inflammation-modulating activities.

The present invention also relates to a process for obtaining live, culture-maintained mammalian cells as these have been described in detail above. The process comprises the following stages:
tissue slices are obtained from functional mammalian tissue by known methods;
the thereby obtained tissue slices are maintained ex vivo on porous membranes having a pore size ≧0.02μ at an acceptable pH-value in the presence of a culture gas in culture in an appropriate culture medium in such a manner that the inter-neighbour relationships and the signal-transduction between cells are retained.

The present invention further concerns a process for the culture of ex vivo maintained live mammalian cells as described in detail above. This process comprises the stages in which mammalian tissue slices, prepared by a known method on porous membranes having a pore size ≧0.02 μm at an acceptable pH-value in the presence of a culture gas in culture in an appropriate culture medium, are maintained in such a manner that the inter-neighbour relationships and the signal-transduction between cells are retained.

The present invention further relates to a process for the induction of inflammatory processes on ex vivo maintained live mammalian cells. This process comprises the following stages:
tissue slices are obtained from functional mammalian tissue by known methods;
the thereby obtained tissue slices are maintained ex vivo on porous membranes having a pore size ≧0.02 μm at an acceptable pH-value in the presence of a culture gas in culture in an appropriate culture medium in such a manner that the inter-neighbour relationships and the signal-transduction between cells are retained; and
the cells are subjected to the action of at least one pro-inflammatory carrier for a transient period of time.

The present invention also concerns a process for the induction of inflammatory processes on ex vivo maintained live mammalian cells. This process comprises the following stages:
tissue slices are obtained from functional mammalian tissue by known methods;
The thereby obtained tissue slices are maintained ex vivo on porous membranes having a pore size ≧0.02μ at an acceptable pH-value in the presence of a culture gas in culture in an appropriate culture medium in such a manner that the inter-neighbour relationships and the signal-transduction between cells are retained; and
the cells are subjected to the action of at least one inflammogen for a transient period of time.

One or more pro-inflammatory carriers or one or more inflammogens can be used individually or in groups or as a number of them in each of the above-mentioned processes without causing any restrictions according to the present invention.

According to preferred embodiments of the present invention the live mammalian cells are subjected ex vivo in culture to the precise action of a substance that is selected from the group of pro-inflammatory carriers and inflammogens over a transient period of time. Thereby, still further preferred embodiments of the invention are to be considered wherein in one of the afore-mentioned processes the pro-inflammatory carrier(s) is/are selected from the group of pro-inflammatory proteins and peptides, preferably from the group of cytokins, and more preferably from the group of CSF-proteins and interferons, and still more preferably from the group comprising GM-CSF, G-CSF and M-CSF and IFNy. In the other afore-mentioned process, the inflammogen(s) is/are especially preferably selected from the endotoxin group, more preferably from the group of lipo-polysaccharides (LPS), still more preferably from the group of bacterial lipo-polysaccharides. Further examples of inflammogens belonging to the endotoxin group are the peptido-glycanes, lipo-teichonic acids, mannose-rich glycanes, flagellin, pillin, non-methylated cytosine-guanine dinucleotides, bacterial DNA, N-formyl-methionine, double-branched RNA, lipo-teichinic acid/glyco-lipids/zymosane from yeast cells, phosphoryl choline and other lipids from bacterial membranes.

The person skilled in the art can determine, or knows on the basis of his experience, the period of contact time of the cells with the selected material(s) that the contact should take for orientating individual tests in the above-mentioned processes. The transient period of time in a case where several materials are used side-by-side or consecutively can be the same or can vary for different substances. The period of time can also vary depending upon the type of mammalian cell used (for example, mouse cell or human cell), or upon the type of cell used (for example, cells from the CNS or cerebral cortex cells). According to the present invention, it is especially preferred if the period of transit time taken where at least one pro-inflammatory carrier is used is in the range of 10 minutes and 7 days, preferably within the range of 15 min. to 4 days, more preferably within the range of 30 min. to 2 days, and/or that the transient period of time taken when at least one inflammogen is used is within the range of 1 day and 14 days, preferably within the range of 1 day to 4 days, and more preferably within the range of 2 days and 3 days.

In all processes according to the present invention, that is to say in the processes which are concerned with the production of ex vivo maintained, live mammalian cells according to the present invention, with the cultivation of the ex vivo maintained live cells according to the present invention and with the induction of inflammatory processes on the live, ex vivo maintained mammalian cells, porous membranes are used that have a pore size ≧0.02 μm. In a preferred embodiment of the present invention, such membranes are permeable to normal culture media including those used in the process of the present invention, and further preferably are also permeable to gases such, for example, as culture gases used in connection with the cultivation of cells.

Still more preferably, membranes according to the invention are those that are also permeable to materials, even oligomeric or polymeric materials that are used in culture medium. These correspond, for example, to a preferred embodiment of the present invention that membranes used for the culture of mammalian cells that are maintained ex vivo are also permeable to at least one pro-inflammatory carrier and/or to at least one inflammogen such as those which are used according to the present invention. With membranes that are permeable to one or more of the mentioned substances, especially good culture results and test-results as well as high throughput rates in the process of the present invention are obtainable.

In particularly preferred embodiments of the present invention, the membranes used have a pore size within the range of 0.02 to 10 μm, still more preferably a pore size within the range of 0.1 to 1 μm and most preferably a pore size within the range of 0.2 to 0.6 μm, for example a pore size of 0.2 or 0.4 μm.

In all of the above-described processes of the invention, the live ex vivo mammalian cells in culture are maintained in an appropriate culture medium. Within this culture medium, the live ex vivo maintained mammalian cells are virtually completely surrounded (insofar as they are not surrounded by other, possibly similar cells) by synthetic media that are mainly liquid, but possibly may also be solid, dissolved and/or gaseous—(but also in gas dissolved) components, which can continuously provide them with components that are essential or useful for specific functions, especially nutrients, trace elements vitamins, materials that maintain the pH-value, buffers, supporting materials for specific cell functions, liberating or modulating materials, antibiotics, and other substances. Culture media that are known to persons skilled in the art can be used without restriction. Such culture media are, for example, those referred to in Dulbecco's Modified Eagle's Medium (DMEM), F12, Neurobasal Medium, MEM, PRMI, (RPMI) 1640, etc. Especially preferred, since they are well or very well suited for the cultivation of the cells provided by the processes of the present invention, for the maintenance of the cells produced by the processes of the present invention, for the testing of the cells produced by the process of the present invention, or for the use of the cells produced by the processes of the present invention, are DMEM/F12 (as a mixture in a volume ratio of 1:1) used as the culture medium. A culture medium can be used individually, or several culture media can be used. This refers as much to a specific stage in the cultivation, maintenance, testing and/or use of the cells, as to a series of several parallel or consecutive procedures in the process, for which the culture medium or media can be used. The culture medium or media can contain known additives that can include but are not restricted to sera (e.g. foetal cattle serum, cattle serum, horse serum, B27, N2), antioxidants such as ascorbic acid, vitamin E, etc.

In the above-mentioned processes of the present invention the ex vivo maintained mammalian cells are maintained in the culture medium at a physiologically acceptable pH-value, that is to say, a pH-value that is tolerable to the cells from a physiological point of view, if not indeed beneficial or promotional for achieving the aims of the invention. In a particularly preferred form of the process of the present invention, the pH-value is a pH-value that conforms to the cellular environment in which the cells naturally reside, or in which they can advantageously be maintained. Especially favourably, the physiologically acceptable pH-value in the process of the present invention lies within the range of 6.5 to 8, more preferably within the range of 6.9 to 7.6, more preferably within the range of 7.3 to 7.5, for example at 7.4.

According to the present invention, the mammalian cells are maintained in the presence of a culture gas in the culture medium. As the culture gas, any gas can be used that is known to the person skilled in the art for the maintenance of mammalian cells ex vivo in culture media and/or to a person skilled in the art who is familiar with the procedures of the present invention for the maintenance, cultivation and induction of inflammatory processes. As examples of gases in the presence of which mammalian cells can be maintained or cultivated in culture media and/or in the presence of which inflammatory processes can be initiated, are such as those selected from the group comprising air (by which is meant not only atmospheric air with its natural composition [essentially, 78.1% by volume of nitrogen, 21.0% by vol. of oxygen, 0.9% by vol. of argon and trace amounts of carbon dioxide], but also other similarly combined gas mixtures, possibly also purified air [“pure air”] and air that has been freed from undesirable components, oxygen, nitrogen, carbon dioxide, air that has been enriched with oxygen, nitrogen or carbon dioxide, etc. According to the present invention, the preferred culture gas used is pure air, preferably pure air with a raised content of CO₂, more preferably pure air with a CO₂ content within the range of 1 to 5% by vol., still more preferably pure air with a CO₂ content within the range of 2 to 3.5% by vol. Cells that are maintained in the presence of the above-mentioned preferred culture gases demonstrate, according to the present invention, particularly good vitality and thus are especially stable ex vivo model systems for the testing of active components of pharmaceutical preparations in respect of their neuro-protective and inflammation-modulating activity.

The above-mentioned procedures can, according to the present invention, be carried out with the use of live cells maintained ex vivo on porous membranes, obtained from any desired mammalian tissues or organs, preferably from tissues or organs of the mammalian nervous system. Examples of such cells are the cells of any mammal or mammalian animal species such, for example, as cells from the guinea pig, mouse, rat, dog, cat or monkey and in preferred embodiments cells of mice, rats or humans, and especially preferably human cells. Human cells are particularly preferred since they provide the best results in respect of intended applications for the vital tissue model, such as those described in detail below.

In the above-mentioned processes of the present invention, the mammalian cells used can be any cells from mammalian tissues or organs. According to the present invention, the preferred cells used are, however, obtained from the mammalian nervous system, and more preferably selected from the group comprising the cells of the central nervous system (CNS), cells from the spinal cord, cells from the hippocampus and cells from the cerebral cortex. Especially preferred are cells from the hippocampus and cells from the cerebral cortex because of their good suitability for the purposes of the aims of the present invention. Such cells are readily available via the above-described methods. The cells used in the processes of the present invention are obtained as bundles of cells in the form of tissue slices of a thickness appropriate to their culture such, for example, as a thickness of 1 to 1000 μm, preferably a thickness of 10 to 800 μm, more preferably a thickness of 100 to 600 μm, for example a thickness of 350 to 450 μm, and advantageously a thickness of 400 μm.

In the process according to the present invention for the induction of inflammatory processes, in the especially preferred embodiments, the inflammogen used comprises at least one, preferably a bacterial, lipo-polysaccharide (LPS) in combination with one or more pro-inflammatory proteins and/or one or more peptides. In these combinations used according to the process the protein(s) and/or peptide(s) is/are more preferably selected from the group of cytokines, more preferably from the group comprising CSF-proteins and interferons, and still more preferably from the group comprising GM-CSF, G-CSF and M-CSF and IFNy.

According to another preferred form of the process, the inflammatory processes are induced by cells, cellular lysates or particles of cells of any kind, specifically cells from the immune system such, for example, as T-lymphocytes, or else by bacteria/moulds, as the provider of pro-inflammatory cytokins, in combination with a lipo-polysaccharide (LPS) or another endotoxin.

The result obtained from the action of one or more substances from the group of inflammogens and pro-inflammatory carrier materials on the live mammalian cells obtained from tissues or organs of a mammal and that are maintained ex vivo in accordance with the processes of the present invention, especially such obtained from the mammalian nervous system, is the manifestation of inflamed processes which, through the maximum activation of the microglia, is characterised by phagocytes. For the purposes of testing, the aim is, and for the model characteristics it is particularly preferred, to obtain a manifestation of inflammatory processes that cause the presence of degenerative end-products, since it is the efficacy of new substances in regard to such degenerative end-products of inflammatory procedures in such cells as models that is intended to be tested.

Within the scope of the present invention for the induction of inflammatory processes, an equally preferred variation to the procedure is to culture the live mammalian cells maintained ex vivo together with cells and/or tissue pieces of other origins, especially preferred being to culture these together simultaneously. The method and type of cells and/or tissue pieces of other origins that are appropriate for use with such preferred procedures, are, according to the present invention, not restricted and can be selected by the person skilled in the art on the basis of his experience, for achieving the aims of the present invention. Particularly preferred are the cells and/or tissue pieces of mammalian cells of other origins and/or mammalian tissue pieces from other mammals than those referred to above, preferably human cells and/or human tissue pieces.

The live mammalian cells maintained ex vivo that were obtained from mammalian tissues and/or organs, especially from tissues and/or organs in the mammalian nervous system, can be used for any purpose known to be appropriate by the person skilled in the art on the basis of his expertise. Especially preferred, within the context of the present invention, is the use of these cells that has been shown to be effective for medical interventions. The medical applications themselves, as the person skilled in the art acknowledges, are wide-reaching and can encompass the field of preventive medicine and/or the therapy of human and animal diseases.

According to a further, especially preferred form of the process, the live, mammalian cells maintained ex vivo that have been derived from mammalian tissues and/or organs, especially from tissues and/or organs of the mammalian nervous system, are used for the ex vivo examination of inflammatory processes, especially for testing pharmaceutical preparations, more preferably for testing the neuro-protective and inflammation-modulating efficacy of pharmaceutical preparations. In this connection, the cells that correspond to one of the above-mentioned requirements for optimum model systems and, with higher throughputs of test substances, permit a similarly good predictability of the efficacy of the substances as do those having far greater time and cost-expenditures associated with the use of whole animal models.

In further preferred forms of the processes of the present invention, the live mammalian cells, maintained ex vivo according to the present invention and that are derived from mammalian tissues and/or organs, especially from tissues and/or organs of the mammalian nervous system are used with excellent predictability results in model tests of the chemical, biological and/or physical effects on the physiological or patho-physiological processes. The following are the especially preferred areas of application in this context according to the present invention:
application in model tests of the chemical, biological and/or physical efficacy in respect of the physiological or patho-physiological processes in mammalian tissues, preferably of the central nervous system (CNS);
application in the target-identification and target-validation for the identification and validation of diagnostic markers and for the development of diagnostic tools for the early identification or acute diagnosis of neuro-degenerative diseases;
application in the clarification of physiological, preferably the patho-physiological activity mechanisms in neuro-degenerative diseases, especially preferably in inflammatory diseases, in Morbus Alzheimer disease, in Morbus Parkinsons disease, in multiple sclerosis, in ALS, in Morbus Huntington disease and in proliferations of the CNS that are associated with inflammatory processes;
application in the screening of active components, in the identification of active ingredients and for validation including the use in toxicity assays;
application in the development of preventive or therapeutically effective substances, preferably of medicines and, most especially preferably, in the development of medicines used for the treatment of inflammatory processes in general, and of diseases of the nervous system.

It has been shown to be especially advantageous and, therefore, most especially preferred, that the above-mentioned areas of application have, without exception, permitted mammalian cells derived from mammalian tissues or organs, especially from tissues or organs of the mammalian nervous system, that are maintained ex vivo in culture in accordance with the above detailed description, to be used together with automated instruments. Thus, in a surprisingly simple and effective manner, a higher throughput of test substances with similarly good predictability in respect of the effectiveness of substances, was possible here as with the use of the far greater time- and cost-expenditure associated with the use of the whole animal model. The automated instruments used in the above-detailed areas of application are known as such to the person skilled in the art; they specifically include the automated platform for the cultivation of organo-typical cultures, that is known by the trade name “Telomics Robotic™”.

The process covered by the present invention for testing the anti-inflammatory efficacy of a chemical substance, is associated with special and thus unexpected advantages in contrast to the prior art, and comprises the following stages:
obtaining tissue slices from functional mammalian tissue, especially from mammalian brain tissue;
culture of the tissue slices obtained ex vivo on porous membranes with a pore size of ≧0.02 μm at a physiologically acceptable pH-value in the presence of a culture gas in culture in an appropriate culture medium in such a manner as to ensure that the inter-neighbour relationships and the signal transduction between cells are retained, and with retention of the tissue culture surrounding the live mammalian cells;
application of at least one pro-inflammatory carrier onto the tissue culture;
application onto the tissue culture of at least one chemical substance whose anti-inflammatory efficiency is to be tested;
application onto the tissue culture of at least one reagent for testing anti-inflammatory efficiency while eliciting a quantifiable confirmation reaction; and
quantification of the confirmation reaction on the basis of the product obtained as a result of the confirmation reaction.

The procedural stages in the process to be carried out are in part in accord with the process described in detail above, for example with the above described process for the ex vivo maintenance of live mammalian cells in accordance with the present invention, for the culture ex vivo of the live mammalian cells maintained according to the present invention, and for the induction of an inflammatory process on the live mammalian cells of the present invention that are maintained ex vivo in culture. Therefore, the description of the stages of this process and their preferred forms of procedure (the preferred porosity and pore size of the membranes used for the culture, the physiologically acceptable pH-value of culture media and its preferred forms of procedure, the presence of a culture gas and the preferred forms of its use, the preferred mammalian cells, the preferred origin of the cells from tissues or organs of the nervous system and their preferred form of handling) can be referenced to the above description and to the preferred embodiments.

In further preferred embodiments for carrying out the test processes according to the present invention, the pro-inflammatory carrier(s) is/are selected from the group of pro-inflammatory proteins and peptides, preferably from the group of cytokins, more preferably from the group of CSF-proteins and interferons, and still more preferably from the group comprising GM-CSF, G-CSF and M-CSF and IFNy. According to the present invention, it is also preferred if the form of procedure in the test process referred to above, consists in allowing at least one pro-inflammatory carrier to act upon the tissue culture for a transient period of time, preferably where the transient period of time for at least one of the pro-inflammatory carriers is within the range of 10 minutes to 7 days, more preferably within the range of 15 min to 4 days, and still more preferably within the range of 30 min to 2 days.

According to a further, equally preferred embodiment of the test process that is either separated from the above-mentioned permitted action of the pro-inflammatory carrier or is carried out simultaneously with it, at least one inflammogen is allowed to act on the tissue culture either simultaneously to or at a separate time from the application of at least one pro-inflammatory carrier on the tissue culture, preferably wherein at least one inflammogen is allowed to act upon the tissue culture after the application of at least one pro-inflammatory carrier. More preferably, the inflammogen(s) is/are selected from the group of endotoxins, and still more preferably from the group of lipo-polysaccharides (LPS). It is still further preferred if at least one inflammogen, advantageously at least one lipo-polysaccharide, and still more preferably if at least one bacterial lipo-polysaccharide is allowed to act upon the tissue culture. Particular advantage is obtained when at least one inflammogen is allowed to act upon the tissue culture over a transient period of time. Preferably, the transient period of time for at least one inflammogen lies within the range of 1 day to 14 days, more preferably within the range of 1 day to 4 days and still more preferably within the range of 2 days to 3 days.

According to the present invention, a successful and therefore a preferred embodiment of the invention is achieved when in the test procedure for the anti-inflammatory effectiveness of a chemical substance, the inflammogen used is a lipo-polysaccharide (preferably a bacterial lipo-polysaccharide) (LPS) in combination with one or more pro-inflammatory proteins and/or peptides.

According to the possibilities provided by the specially preferred embodiment of the present invention for carrying out the test procedures according to the present invention, at least one reagent is used that is introduced onto the tissue culture for testing the anti-inflammatory effectiveness that is a reagent that allows physical measurements to be carried out, preferably a reagent that allows optical measurements to be carried out, more preferably a staining reagent that is accessible for optical measuring methods, still more preferably a staining reagent that is accessible for fluorescence measurements. This allows a rapid and reliable quantitative identification of the anti-inflammatory effectiveness to be carried out, which has the additional advantage that automated measurements can be undertaken. In a surprising and advantageous manner, this allows the test procedure to be undertaken advantageously through the utilization of automated laboratory instrumentation. Thus, with the higher throughput of test substances, a similarly good predictability of the effectiveness of the substances is achievable, as with the much more time and cost-expenditure use of whole animal models. In this way, the test procedures of the present invention, also for screening processes, make a larger number of potentially active substances available.

The invention is described further by way of an example of a form of the process, which is not restricted thereto, from which the person skilled in the art can especially identify the advantages of the preferred embodiments of the invention.

In the case of organo-typical cultures, the process is concerned with 400 μm-thick slices obtained from live mammalian tissue such, for example, as tissue from the hippocampus sector of the cortex, and that are cultivated on porous membranes. The mass-cultivation and excellent availability guarantee a good throughput of test substances.

At the same time, the relationships between neighbouring cells and the signal transduction between the various cell types in the brain, that are also present in the tissue slices, are retained, which can be demonstrated by means of functional analyses. It can, therefore, be assumed that the inter-communication between cells in organo-typical cultures reflects the relationships in the live organism or, at least, re-capitulates those very well. Thereby, an excellent degree of transferability of the results obtained from these cultures, to animal models is achieved. It can, therefore, be expected that there is a good degree of conformity of the active, neuro-protective substances in organo-typical tissue culture models with those in animal models, which was able to be confirmed by means of the validation of known inflammation-inhibitors through our assays (see the examples and the FIG. 2).

Reference has already been made to the fact that the microglia play a central role in inflammatory processes in the brain under physiological as well as patho-physiological conditions. After preparation of the brain tissue slices, the endogen, i.e. the microglia residing in the tissue, are activated. After a phase of recovery and levelling up of the tissue lasting about 5 to 6 days that also proceeds together with removal of the damaged neurons, the microglia cells return to their state of rest which is recognised by a branched morphology (Czapiga M., Colton C. A. (1999): function of microglia in organo-typic slice cultures. J. Neirosci. Res. 56., 644-651; author's observations). This so-called resting microglia is a characteristic of the normal state of healthy, adult brain tissue. With the addition of, for example, bacterial LPS, a (partial) activation of the microglia takes place that does not lead to a degeneration of the neurons.

Thereby, the expression of MHC—(major histo-compatibility complex)—proteins seems to be particularly correlated with the activation of the microglia to a phagocytotic, round phenotype which is co-localised with lesions in the injured brain. Neo-natal microglia react in the same form and manner to the pro-inflammatory cytokins GM-CSF and M-CSF, i.e. by changing their morphology and phenotype, and furthermore, are stimulated to undergo division. GM-CSF particularly promotes the construction of a round, migratory phenotype which, in addition, is prepared for the presentation of antigens. This can be confirmed through the expression of the “major histo-compatibility complex-(MHC)-Class II-Antigens (also known as Ox-6 markers), which are induced by GM-CSF (Schermer C., Humpel C., 2002; Granulocyte Macrophage Colony-Stimulating Factor (GM-CSF) activates microglia in rat cortex organotypic brain slices. Neurosci. Lett. 328: 180-184; Re F. Belyanskaya , S. L., Riese R. J., Cipriani B., Fischer F. R., Granucci F., Ricciardi-Gastagnoli P., Brosnan C., Stern L. J., Stromionger J. L., Santambrogio L. (2002). Granulocyte-macrophage colony-stimulating factor induces an expression program in neo-natal microglia that primes them for antigen presentation; J. Immunol., No. 1, 169: 2264-2273). It was interesting to show that intact neurons are able to suppress the induction capacity of MHC-class II antigens on the neighbouring microglia cells (Neumann H. (200111): Control of glial immune function by neurons. Glia 36: 191-199). It is possible that this local inhibition is lifted by priming of the microglia with GM-CSF, so that pathological activation of the microglia can be activated by a subsequent or parallel incubation with 200 μm/ml of LPS within a period of 48 hours (see, FIG. 1). This activation can be detected with the antibody Ox 6 thor's unpublished data). Also integrines (detectable by the antibody C 42) or the inducible NO syntases, are frequently used as markers for activated microglia, although their interpretation capacity is less apparent. An expression of these markers that indicates activation of the microglia and accompanies a clear change in the morphology, has already taken place with the simple addition of the pro-inflammatory cytokins or inflammogens (e.g. Schermer C., Humpel C. (2002): Granulocyte macrophage-colony stimulating factor activates microglia in rat cortex organotypic brain slices. Neurosci. Lett. 328: 180-184; authors data). These stages in the microglia activation, however, does not lead to any cell injury or to neuro-degeneration, which explains why it is not used in our assays as the parameter of choice and is unsuitable for testing potential neuro-protective reagents.

The activation of the microglia to phagocytotic phenotype possibly even mirrors a “therapeutic” function of the microglia, which is essential for the removal of pathogens or cell debris. This function of the microglia should, therefore, be retained. An over-activation of the microglia, however, apparently also leads to the degeneration of the tissue and neurons, and thus to pathological functions. These pathological functions of the microglia should be controlled or inhibited as far as possible, without them thereby losing their therapeutic functions. So far, the therapeutic activation cannot be distinguished from the pathological activation of the microglia by means of a change in the marker profile of the microglia, possibly because these conditions or functions overlap or become coupled together in pathological situations. For this reason, the active substances are tested in an assay with the use of the injury or degeneration of the tissue itself as the measure of pathological functions of the microglia (see, FIGS. 1 and 2). Only in this manner can it be confirmed whether the active substances actually have a specific influence upon the pathological functions of the microglia and thereby inhibit degeneration of the tissues. Furthermore, there is the possibility that the active substances found within this screen “uncouple” the pathological from the therapeutic functions, thus modulating the microglial activation instead of suppressing it in a non-specific manner.

Active ingredients that fulfil these profile requirements are the highly interesting reagent candidates that could specifically suppress injurious inflammation reactions of the microglia during the progress of neurological conditions such, for example, as stroke, Morbus Alzheimer and many other conditions. The search for such reagents is frequently described in the literature as being desirable, although a model for the systematic screening of such reagents has not been made available to date.

The present invention is further described by way of the following examples. These examples relate to preferred forms of procedure according to the present invention, including the currently known best forms of the process of the invention, although the invention is not restricted to the processes of the examples disclosed here.

### EXAMPLES

### Example 1

For the preparation of organo-typical cultures of the hippocampus, 7 to 8 day-old Sprague-Dawley rats were used. From the isolated hippocampus of these animals ca. 400 μm thick slices were prepared, each approximately comprising 20 cell layers. 5 to 6 slices were placed onto a membrane insert (Millicel; the firm Millipore) having an average pore size of 0.4 μm, and in such a manner that the slices did not touch one another. The slices were able to be cultured in 6-pore culture plates for up to 3 weeks through changing the medium below the membrane. The injury to the cells arising from the preparation, especially on the slice surfaces, led to temporary activation of the microglia residing in the slices, that died away after 5 to 6 days. This took place together with the elimination of the cell debris by the now phagocytic microglia. During this first culture period the slices became substantially flatter, which was observable in penetrating light through the increase in transparency of the slices. Membranes that had a majority of transparent slices elicited almost no injury or neuro-degeneration (<1%) after one week and could, therefore, be used as starting material for an assay.

### Example 2

When the resting microglia in this culture were activated by the pro-inflammatory cytokin GM-CSF or M-CSF, a pathological (over-) activation of the microglia through a subsequent or parallel incubation with 200 μm/ml of LPS within 48 hours took place, which also resulted in a dramatic degeneration of neurons, (see FIG. 1). Activated microglia also reacted to LPS (over the 48 hours) with the cytokin M-CSF (10 ηg/ml), with increased neuro-toxicity; however, the resulting degeneration of the tissue in this case was less precipitous, (see FIG. 1). All the incubations were carried out in a medium that contained a minimum of 50% of essential medium/HEPES, 25% of Hank's balanced salt solution and 25% of heat-inactivated horse serum.

The degeneration of the tissue was rendered visible by means of a propidium iodide-fluorescence of the cells, which, because of the injury sustained, could no longer prevent, due to intact cell membranes, diffusion into the cell nucleus and staining of the DNA. Consequently, the red fluorescence above a threshold value was evaluated as the signal for the degeneration of cells. The degree of degeneration per slice was expressed as the percentage of the total surface that the red values lay above the threshold. By means of this quantification of the cell injury it was apparent that LPS alone did not lead to degeneration, although the combination of cytokin priming and LPS did (see FIG. 1).

### Example 3

A further pro-inflammatory cytokin that activates the mictroglia and can induce the MHC expression is IFNy. With cultivation of slices with 30 ηg/ml of IFNy together with 200 ηg/ml of LPS over 48 hours, a very strong degeneration of the tissue was induced (data not indicated).

This degeneration of neuronal tissue by the over-activation of the microglia is, therefore, strangely due to inflammatory processes and not to a direct neuro-degenerative activity of LPS or GM-CSF. For that reason, anti-inflammatory reagents can work in such assays for the complete protection of the neural tissue, which could be shown to be the case, for example, for acetyl salicylic acid and dexamethasone (see FIG. 2). Here, both reagents were incubated over 48 hours with LPS and GM-CSF. Thus, for working in this assay, both steroidal as well as non-steroidal reagents were used. The protective activity declined gradually with reducing concentrations of reagent, which is the reason why weaker reagents could also be recognised. Thus, for example, a protective activity by iboprufen could also be shown to exist (data not disclosed). Furthermore, a protective activity of a plant extract could be demonstrated, which correlated very well with in vivo investigations. A high degree of transferability of the results onto the in vivo conditions could, therefore, already at the experimental level, be underpinned. Since substances work, therefore, in this new model, for the inflammation-activated neuro-degeneration, further testing in the animal world seems to be especially promising of success.

### INTERPRETATION OF THE FIGURES

FIG. 1: From inflammation of provoked degeneration of neural tissue in organo-typical cultures by the combination of microglia-activating cytokins with the endotoxin LPS.

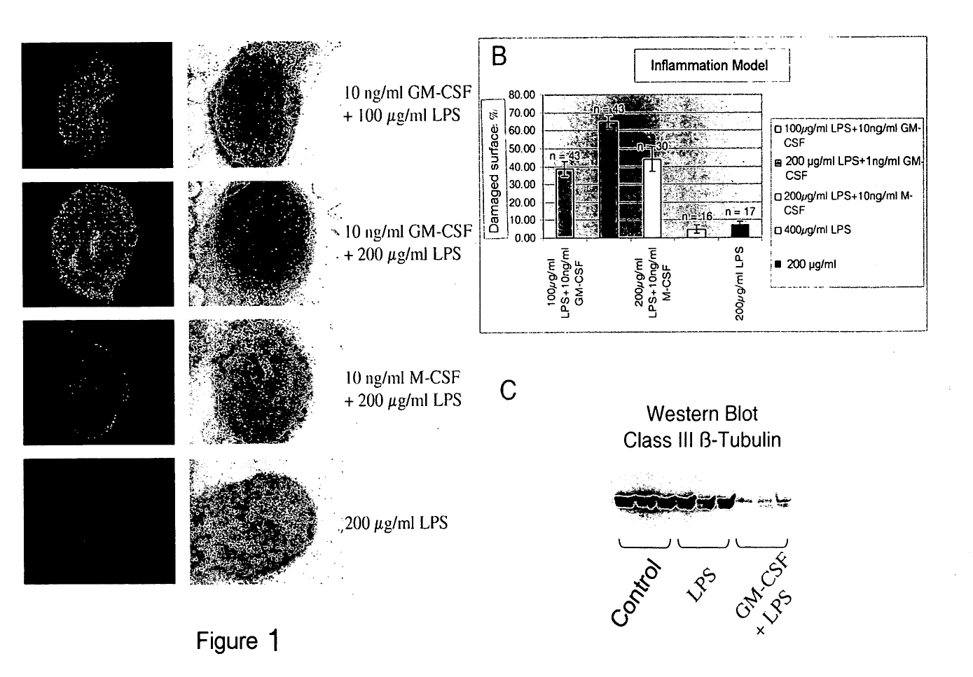

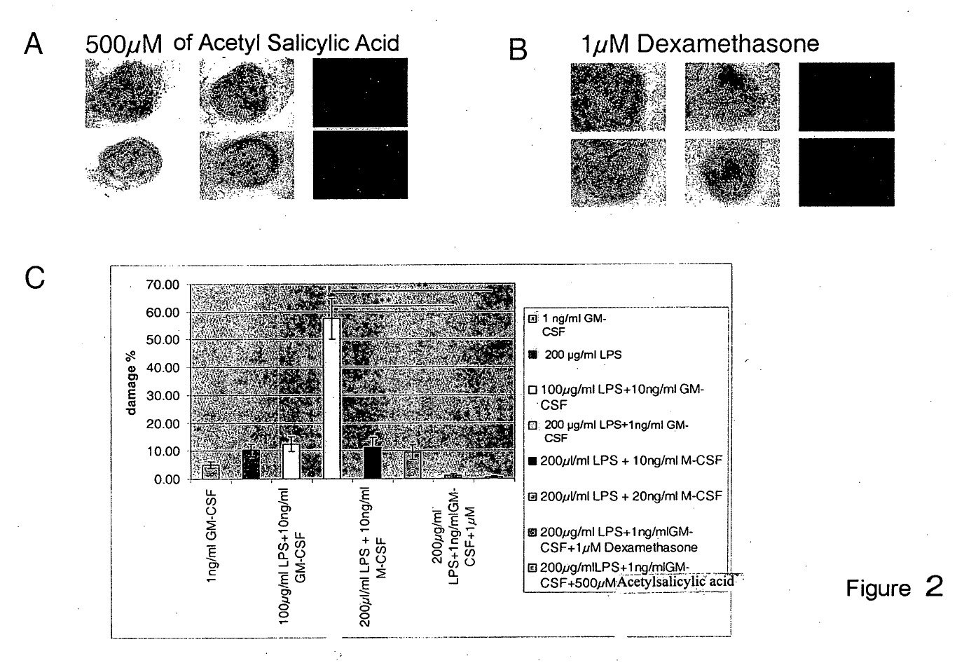

Organo-typical cultures were incubated with LPS either alone or in combination with the pro-inflammatory cytokins GM-CSF and M-CSF over 48 hours. The injury to the tissue (cell mortality) was established by means of propidium iodide staining (A) and quantified by means of an automated picture analysis (B). By means of a Western Blot on the cultures after the optical evaluation, with the help of a neuronal marker (class III β-Tubulin), it could be shown that the over-activation of the microglia inside the tissue also leads to a neuro-degeneration, i.e. to the necrosis of the neurons (C). LPS alone (or GM-CSF; not shown), did not lead to any detectable injury or to any neuro-degeneration that could be confirmed.

FIG. 2: Degeneration with GM-CSF in combination with LPS and protection of the tissue by two different anti-inflammatory reagents.

Two representative examples of propidium iodide staining following incubation of the organo-typical cultures with 1 ηg/ml of GM-CSF and 200 ηg/ml of LPS, which, at the same time, are protected by the anti-inflammatory reagents acetyl salicylic acid (A) or dexamethazone (B) against expected injury. By quantification of the results (C) a maximum injury of the cultures through the combination of 1 ηg/ml of GM-CSF with 200 ηg/ml of LPS was elicited. This combination was selected, also for the experiments with anti-inflammatory substances, because it could almost completely prevent degeneration of the tissue. Similar results were achieved with pre-incubation of 10 ηg/ml GM-CSF over one hour followed by 48 hours incubation with LPS, with or without reagents (data not disclosed).

1.-50. (canceled)

51. A plurality of live cells obtained from a mammalian tissue or organ, wherein the cells are maintained ex vivo on a porous membrane having a pore size of ≧0.02 μm at a physiologically acceptable pH, in the presence of a culture gas, in culture and in a culture medium and wherein inter-neighbour relationships and signal transduction between the cells are retained.

52. The plurality of live cells of claim 51, wherein the membrane is pervious to the culture medium and the culture gas.

53. The plurality of live cells of claim 52, wherein the membrane has a pore size of from 0.02 μm to 10 μm.

54. The plurality of live cells of claim 51, wherein the physiologically acceptable pH is from 6.5 to 8.

55. The plurality of live cells of claim 51, wherein the culture gas comprises pure air.

56. The plurality of live cells of claim 55, wherein the pure air has a raised CO₂ content of from 1% to 5% by volume.

57. The plurality of live cells of claim 51, wherein the cells are selected from one or more of rat cells, mouse cells and human cells.

58. The plurality of live cells of claim 51, wherein the cells comprise cells from a nervous system of a mammal.

59. The plurality of live cells of claim 58, wherein the cells comprise at least one of cells of a CNS, cells from a spinal cord, cells from a hippocampus and cells from a cerebral cortex.

60. The plurality of live cells of claim 51, wherein the cells have been subjected over a transient period of time to the action of at least one substance selected from pro-inflammatory carriers and inflammogens.

61. The plurality of live cells of claim 60, wherein the membrane is pervious to the at least one substance.

62. The plurality of live cells of claim 51, wherein the cells are from a nervous system of a at least one of a rat, a mouse and a human, the porous membrane has a pore size of from 0.02 μm to 10 μm, the pH is from 6.5 to 8 and the culture gas comprises pure air.

63. The plurality of live cells of claim 51, wherein the cells are from a nervous system of a at least one of a rat, a mouse and a human and comprise at least one of cells of a CNS, cells from a spinal cord, cells from a hippocampus and cells from a cerebral cortex, the porous membrane has a pore size of from 0.1 μm to 1 μm, the pH is from 6.9 to 7.6 and the culture gas comprises pure air having a raised CO₂ content of from 1% to 5% by volume.

64. The plurality of live cells of claim 63, wherein the porous membrane has a pore size of from 0.2 μm to 0.6 μm, the pH is from 7.3 to 7.5 and the culture gas comprises pure air having a raised CO₂ content of from 2% to 3.5% by volume.

65. A process for obtaining and/or culturing the plurality of live cells of claim 51, wherein the process comprises maintaining ex vivo tissue slices obtained from functional mammalian tissue on a porous membrane having a pore size of ≧0.02 μm at a physiologically acceptable pH in the presence of a culture gas in culture in a culture medium in such a manner that inter-neighbour relationships and signal transduction between the cells are retained.

66. A process for inducing inflammatory processes on live mammalian cells maintained ex vivo, wherein the process comprises maintaining ex vivo tissue slices obtained from functional mammalian tissue on a porous membrane having a pore size of ≧0.02 μm at a physiologically acceptable pH in the presence of a culture gas in culture in a culture medium in such a manner that inter-neighbour relationships and signal transduction between the cells are retained and subjecting the cells to an action of at least one pro-inflammatory messenger over a transient period of time.

67. The process of claim 66, wherein the at least one pro-inflammatory messenger is selected from pro-inflammatory proteins and peptides.

68. The process of claim 67, wherein the at least one pro-inflammatory messenger is selected from cytokins.

69. The process of claim 66, wherein the transient period of time is from 10 minutes to 7 days.

70. The process of claim 66, wherein the process manifests inflamed processes in the form of activation of microglia to phagocytes.

71. The process of claim 66, wherein the mammalian cells are cultivated together with at least one of cells and pieces of tissue that are derived from other sources.

72. A process for inducing inflammatory processes on live mammalian cells maintained ex vivo, wherein the process comprises maintaining ex vivo tissue slices obtained from functional mammalian tissue on a porous membrane having a pore size of ≧0.02 82 m at a physiologically acceptable pH in the presence of a culture gas in culture in a culture medium in such a manner that inter-neighbour relationships and signal transduction between the cells are retained and subjecting the cells to an action of at least one inflammogen over a transient period of time.

73. The process of claim 72, wherein the at least one inflammogen is selected from endotoxins.

74. The process of claim 72, wherein the transient period of time is from 1 day to 14 days.

75. The process of claim 72, wherein the at least one inflammogen is used in combination with at least one substance selected from pro-inflammatory proteins and peptides.

76. The process of claim 72, wherein the process manifests inflamed processes in the form of activation of microglia towards phagocytes.

77. The process of claim 72, wherein the mammalian cells are cultivated together with at least one of cells and pieces of tissue that are derived from other sources.

78. A method for the ex vivo investigation of inflamed processes, wherein the method comprises using the plurality of live cells of claim 51.

79. A method of investigating chemical, biological and/or physical effects on physiological or patho-physiological processes on a model, wherein the method comprises using the plurality of live cells of claim 51 as the model.

80. A process for determining the anti-inflammatory efficacy of a chemical substance, comprising:
ex vivo cultivation of tissue slices obtained from functional mammalian tissue on porous membranes having a pore size of ≧0.02 μm at a physiologically acceptable pH-value in the presence of a culture gas in culture in a culture medium in such a manner that inter-neighbour relationships and signal transduction between cells are retained, thereby obtaining a tissue culture that comprises live mammalian cells;
application of at least one pro-inflammatory messenger onto the tissue culture;
application of at least one chemical substance whose anti-inflammatory efficacy is to be determined onto the tissue culture;
application of at least one reagent for detecting anti-inflammatory efficacy onto the tissue culture to obtain a quantifiable detection reaction; and
quantification of the detection reaction based on a product obtained from the detection reaction.

81. The process of claim 80, wherein the at least one pro-inflammatory messenger is selected from pro-inflammatory proteins and peptides.

82. The process of claim 80, wherein the at least one pro-inflammatory messenger is allowed to react with the tissue culture for a transient period of time of from 10 minutes to 7 days.

83. The process of claim 80, wherein at least one inflammogen is applied to the tissue culture either simultaneously or separately from the application onto the tissue culture of at least one pro-inflammatory messenger.

84. The process of claim 83 wherein the at least one inflammogen comprises at least one endotoxin.

85. The process of claim 83, wherein the at least one inflammogen is allowed to react with the tissue culture over a transient period of time of from 1 day to 14 days.

86. The process of claim 80, wherein the at least one reagent for detecting anti-inflammatory efficacy on the tissue culture comprises a reagent which permits use of physical measuring methods.

87. The process of claim 80, wherein the process is suitable for use with automated laboratory instrumentation.
